(19) United States
(12) Patent Application Publication
(10) Pub. No.: US 2008/0019941 A1
(43) Pub. Date: Jan. 24, 2008

(54) Methods, systems and reagents for scar reduction

(76) Inventor: Susan J. Drapeau, Cordova, TN (US)

Correspondence Address: FOX ROTHSCHILD, LLP, 997 LENOX DRIVE, LAWRENCEVILLE, NJ, 08648, US

(21) Appl. No.: 11/490,413

(22) Filed: Jul. 20, 2006

(51) Int. Cl. A61K 38/21; A61K 38/18; A61F 13/02

(52) U.S. Cl. 424/85.4; 514/12; 424/448

(57) ABSTRACT

The present invention is directed towards the use and delivery of a therapeutic method and agent to reduce and prevent excessive scarring resulting from cellular contraction and/or excess accumulation of extracellular matrix by inhibition and/or inducement of smooth muscle actin (SMA) and/or tissue growth factor beta (TGF-β) in a tissue, organ or injury site.

### FIELD OF THE INVENTION

The invention relates generally to the use and delivery of a therapeutic agent to both block and induce at various times and places smooth muscle actin (SMA) and/or tissue growth factor beta (TGF-β) to reduce excessive scarring.

### BACKGROUND OF THE INVENTION

Abnormal expression of Tissue Growth Factor (TGF) has been associated with general tissue scarring, tumor-like growths in the skin, and sustained scarring of blood vessels, leading to impaired blood-carrying ability, hypertension, hypertrophy, etc. Also associated with TGF are various diseases caused by vascular endothelial cell proliferation or migration, such as cancer, including dermatofibromas, conditions related to abnormal endothelial cell expression, breast carcinoma desmosplasis, angiolipoma, and angioleiomyoma. Other related conditions include atheroscelrosis and systemic sclerosis, including atherosclerotic plaques, inflammatory bowel disease, Chrohn's disease, angiogenesis, and other proliferative processes which play central roles in atherosclerosis, arthritis, cancer, and other disease states, neovascularization involved in glaucoma, inflammation due to disease or injury, including joint inflammation, tumor growth metastasis, interstitial disease, dermatological diseases, arthritis, including chronic rheumatoid arthritis, arteriosclerosis, diabetes, including diabetic nephropathy, hypertension, and other kidney disorders, and fibrosis resulting from chemotherapy, radiation treatment, dialysis, and allograft and transplant rejection.

There are numerous examples of fibrosis, including the formation of scar tissue following a heart attack, which impairs the ability of the heart to pump. Diabetes frequently causes damage/scarring in the kidneys which leads to a progressive loss of kidney function. Even after surgery, scar tissue can form between internal organs causing contracture, pain, and, in some cases, infertility. Major organs such as the heart, kidney, liver, lung, eye, and skin are prone to chronic scarring, commonly associated with other diseases. Hypertrophic scars (non-malignant tissue bulk) are a common form of fibrosis caused by burns and other trauma. In addition, there are a number of other fibroproliferative disorders such as scleroderma, keloids, and atherosclerosis which are associated respectively with general tissue scarring, tumor-like growths in the skin, or a sustained scarring of blood vessels which impairs blood carrying ability. As TGF is over-expressed in fibrotic disorders, it represents a very specific target for the development of anti-fibrotic therapeutics.

Also associated with tissue scarring due to muscle movement is smooth muscle actin (SMA). Actin is a globular structural protein that polymerizes in a helical fashion to form an actin filament (or microfilament). These form the cytoskeleton—a three-dimensional network inside an eukaryotic cell. Actin filaments are assembled in two general types of structures: bundles and networks and provide mechanical support for the cell, determine the cell shape, enable cell movements (through lamellipodia, filopodia, or pseudopodia); and participate in certain cell junctions, in cytoplasmic streaming and in contraction of the cell during cytokinesis. In muscle cells they play an essential role, along with myosin, in muscle contraction.

Actin, together with myosin filaments, form actomyosin, which provides the mechanism for muscle contraction. Muscular contraction uses the ATP energy transfer molecule for energy. The ATP molecule allows, through hydrolysis, the myosin head to extend up and bind with the actin filament. However ATP is not needed to the attachment of myosin (in muscle it is myosin II) onto the actin filament. The myosin head then releases after moving the actin filament in a relaxing or contracting movement by usage of nucleotide adenosine diphosphate (ADP). Accordingly, in all but three of the ten types of muscles use the movement of actin against myosin to create contraction and relaxation.

By extension contrarily, cellular contraction also plays an important role in wound healing. Although contraction may initially promote healing, it can also lead to significant scarring and a loss of physiological function (see, for example, U.S. Pat. No. 5,741,777). The adverse effects of contraction are particularly severe in surgical and burn patients. In addition, scarring may cause secondary damage to patients that have incurred damage to the spinal cord or other severe trauma.

In view of the foregoing, it can be appreciated that treatment measures are needed to treat and prevent tissue scarring.

### SUMMARY OF THE INVENTION

Accordingly, the present invention introduces the use and delivery of a therapeutic agent to block and/or induce tissue smooth muscle actin (SMA) and/or growth factor beta (TGF-β) in order to reduce excessive scarring.

In an aspect, the present invention is directed to a method of repairing musculoskeletal tissue (including bone, articular cartilage, meniscus, tendon intervertebral disc and especially damaged ligaments) and epithelial tissue by inhibiting contraction of a muscle by administering an effective amount of an inhibitor of smooth muscle actin and/or growth factor beta. Such therapeutic inhibitor agent can be delivered in powder or solution form, and either alone or in combination with a natural or synthetic carrier. The agent can also be delivered alone or in combination with an anti-inflammatory, pain reliever or other therapeutic.

In another aspect, the invention is directed to a procedure for promoting the healing of wounded musculoskeletal tissue (particularly a damaged ligament) in a patient by sequentially inhibiting muscle contraction and then promoting muscle contraction by administering an SMA inhibitor followed by an SMA inducer. The SMA inhibitor should be given at a dosage and for a duration sufficient to promote tissue attachment. The time necessary for attachment to occur will vary from patient to patient, but will typically be between 1 and 10 weeks. The extent to which attachment has occurred may be determined by clinical examination and by diagnostic imaging techniques well known in the art. After attachment, the SMA inducer should be administered for the purpose of causing the tissue to contract and thereby assume a more natural conformation. Among the agents that may be used for inhibiting contraction are PDGF and interferon. Among the agents that may be used to promote contraction is TGF-β (i.e., 100 ng/ml to 500 ug/ml).

In yet an alternative aspect to the above aspect, the present invention is directed to a method of treating a patient for damaged musculoskeletal tissue and epithelial tissue by sequentially promoting muscle contraction and then inhibiting muscle contraction by administering an SMA inducer followed by an SMA inhibitor. Initially, an SMA inducer is injected at the site of tissue damage at a dosage and for a duration sufficient to promote the repair of the tissue. Once tissue repair has been essentially completed, an SMA inhibitor may be administered at the site of the damage to reduce scar formation. Thus, one example of a treatment protocol using this procedure would involve injections of TGF-β at a concentration of between 100 ng/ml and 500 ug/ml at the site of ligament damage, e.g., the knee. After a period of, for example, 4 weeks, injections are made using a comparable concentration of PDGF or an interferon until healing is complete.

The activity of SMA may also be reduced by preventing its expression using an antisense oligonucleotide, particularly an oligonucleotide complementary to the promoter region of the human SMA gene. In most cases, it is expected that administration will be accomplished using local delivery.

Lastly, inducers of SMA may also be administered to a patient for the purpose of enhancing drug absorption. A sufficient dosage should be given to induce endothelial cell contraction. For example, TGF-β at a concentration of 100 ng/ml-500 ug/ml can be co-administered with a second drug either parenterally or intranasally.

The present invention, including its features and advantages, will become more apparent from the following detailed description.

### DETAILED DESCRIPTION

The present invention is directed towards the use and delivery of a therapeutic agent to both block and induce at various times and places smooth muscle actin (SMA) and/or tissue growth factor beta (TGF-β) to reduce excessive scarring by control of muscle contraction.

By way of overview, TGF-β, as a SMA inducer is a polypeptide. Peptides (from the Greek πεπτoç, “digestible”), are the family of short molecules formed from the linking, in a defined order, of various α-amino acids. The link between one amino acid residue and the next is an amide bond, and is sometimes referred to as a peptide bond. Like proteins, peptides are polypeptide molecules. The distinction is that peptides are short and proteins are long. There are several different conventions to determine these, all of which have flaws.

One convention is that those peptide chains that are short enough to be made synthetically from the constituent amino acids are called peptides rather than proteins. However with the advent of better synthetic techniques, peptides as long as hundreds of amino acids can be made, including full proteins like ubiquitin. Native chemical ligation has given access to even longer proteins, and so this convention seems to be outdated.

Another convention places an informal dividing line is at approximately 50 amino acids in length (some people claim shorter lengths). However, this definition is somewhat arbitrary—some peptides such as alzheimer's beta peptide come close and some proteins (such as insulin) are close to the lower limit for proteins. Because of the arbitrary nature of this definition, there is considerable movement within the scientific community to ascribe the more-specific definition that “a peptide is an amino acid molecule without secondary structure; on gaining defined structure, it is a protein.” Thus the same molecule can be either a peptide or a protein depending on its environment, though there are peptides that cannot be proteins.

In any event, Tissue Growth Factor (TGF) is used to describe two classes of polypeptide growth factors, TGFα and TGFβ, which are defined by their ability to cause oncogenic transformation in a specific cell culture system: the growth of treated cells is no longer inhibited by contact between cells, and could progress in soft agar where cells are no longer anchored to a surface. The two classes of TGFs are not related to one another, act through different receptor mechanisms, do not always induce cell division, and are not the only growth factors resulting in cellular transformation.

TGFβ exists in at least three known subtypes in humans, TGFβ1, TGFβ2, and TGFβ3. These are up-regulated in some human cancers, and play crucial roles in tissue regeneration, cell differentiation, and embryonic development. TGFβ1 frequently exerts a growth inhibitory role on epithelial cells, becoming expressed at high levels late in regenerative processes as cell division comes to an end. The TGFβ super-family includes other homologous ligands including activin and bone morphogenetic proteins.

It is known that TGFβ is indicated in the causation of fibrotic conditions. During normal tissue repair, TGFβ production is increased to stimulate the process of repair. When repair is complete, TGFβ production is reduced. If not reduced following normal tissue repair, the increased TGFβ overproduction can result in the development of excess extracellular matrix accumulation and fibrotic conditions. Thus, repeated tissue injury or a defect in TGFβ regulation leading to sustained TGFβ production results in excess accumulation of extracellular matrix (ECM).

As such it should be understood that TGFβ overproduction may result from multiple pathways and require that more than one pathway be inhibited to achieve any clinically significant reduction in scarring due to excess accumulation of ECM and the amelioration of associated disease. Thus optimal therapy of disorders associated with TGFβ overproduction must take into account the multiple pathways of TGFβ production to effectively combat overproduction of TGFβ. Without such multi-faceted strategy, inhibition of one pathway of TGFβ production may be insufficient to block excess accumulation of ECM and can even result in an increase in the levels of TGFβ production by stimulation of one of the alternative pathways for its production.

As used herein “inhibition of TGFβ” includes inhibition of TGFβ activity, for example in causing excess deposition of ECM, as well as inhibition of TGFβ production resulting in overproduction and excess accumulation of ECM, regardless of the mechanism of TGFβ activity or overproduction. This inhibition can be caused directly, e.g. by binding to TGF or its receptors, for example by anti-TGFβ antibodies or TGFβ receptor antagonists, or can be caused indirectly, for example by inhibiting a pathway that results in TGFβ production, such as the renin pathway. Inhibition causes a reduction in the ECM producing activity of TGFβ regardless of the exact mechanism of inhibition.

As used herein a “TGFβ inhibitory agent” is an agent that directly or indirectly inhibits TGFβ binding to its receptors, such as a TGFβ-specific inhibitory agent, or an agent that blocks an alternative pathway of TGFβ production. The agent causes a reduction in the ECM producing activity of TGFβ regardless of the mechanism of its action. The agent can be nucleic acid encoding the TGFβ inhibitory agent such as a cDNA, genomic DNA, or an RNA or DNA encoding TGFβ inhibitory activity such as a TGFβ antisense RNA or DNA.

As used herein, a “TGFβ-specific inhibitory agent” means an agent containing TGFβ inhibiting activity, including agents that bind directly to TGFβ such as anti-TGFβ antibodies, or are a ligand for TGFβ which prevents it from binding to its receptors. A TGFβ-specific inhibiting agent also includes a nucleic acid encoding a particular TGFβ-specific inhibitory agent such as a cDNA, genomic DNA or an RNA or DNA encoding TGF-specific inhibitory activity such as a TGFβ antisense RNA or DNA.

Agents that bind directly to TGFβ are known and include anti-TGFβ antibodies such as anti-TGFβ1 antibodies (Genzyme, Cambridge, Mass.) and antibodies which bind both TGFβ1 and TGFβ2 (see for example, Dasch et al., U.S. Pat. No. 5,571,714), proteoglycans such as decorin, biglycan and fibromodulin, and the nucleic acids encoding such agents.

Antibodies to inhibit TGFβ, renin or other molecules, for use in the present invention, can be prepared according to methods well established in the art, for example by immunization of suitable host animals with the selected antigen, e.g. TGFβ. For descriptions of techniques for obtaining monoclonal antibodies see, e.g. the hybridoma technique of Kohler and Milstein (Nature 256:495-497 (1975)), the human B-cell hybridoma technique (Kosbor et al., Immunol. Today 4:72 (1983); Cole et al., Proc. Nat'l. Acad. Sci. USA, 80:2026-2030 (1983)) and the EBV-hybridoma technique (Cole et al., Monoclonal antibodies and Cancer Therapy, Alan R. Liss, Inc., pp. 77096 (1985)). Such antibodies may be of any immunoglobulin class including IgG, IgM, IgE, IgA, IgD and any subclass thereof. The hybridoma producing the monoclonal antibody may be cultivated in vitro or in vivo. Suitable host animals include, but are not limited to, rabbits, mice, rats, and goats. Various adjuvants may be used to increase the immunological response to the host animal, depending on the host species, including, but not limited to, Freund's (complete and incomplete), mineral gels such as aluminum hydroxide, surface active substances such as pluronic polyols, polyanions, peptides, oil emulsions, keyhole limpit, hemocyanin, dinitrophenol and potentially useful human adjuvants such as BCG (Bacille Calmette-Guerin) and Cornebacterium parvum. Antibodies as used herein includes non-human, chimeric (different species), humanized (see Borrebaeck, Antibody Engineering: A Practical Guide, W. H. Freeman and Co., New York, 1991), human and single-chain antibodies, as well as antibody fragments including but not limited to the F(ab′)2 fragments that can be produced by pepsin digestion of antibody molecules and Fab fragments that can be generated by reducing disulfide bridges of the F(ab′)2 fragments. Alternatively, Fab expression libraries may be constructed (Science 246:1275-1281 (1989)) to permit the rapid and easy identification of monoclonal Fab fragments having the desired specificity.

An indirect TGFβ inhibitor would inhibit the synthesis or secretion of TGFβ or sequester it away from its target cells. Such inhibitors include, but are not limited to, inhibitors of Angiotensin Converting Enzyme (“ACE”), antagonists of the AII receptor such as Losartan™ and Cozar™ (Merck), and aldosterone inhibitors such as Spironolactone™ (Sigma Chemical Co., St. Louis, Mo., Product # S 3378) that would otherwise result in increased TGFβ production.

Also included within the scope of TGFβ inhibitors of the invention are nucleic acids that include antisense oligonucleotides that block the expression of specific genes within cells by binding a complementary messenger RNA (mRNA) and preventing its translation (See review by Wagner, Nature 372:332-335 (1994); and Crooke and Lebleu, Antisense Research and Applications, CRC Press, Boca Raton (1993)). Gene inhibition may be measured by determining the degradation of the target RNA. Antisense DNA and RNA can be prepared by methods known in the art for synthesis of RNA including chemical synthesis such as solid phase phosphoramidite chemical synthesis or in vitro and in vivo transcription of DNA sequences encoding the antisense RNA molecule. The DNA sequences may be incorporated into vectors with RNA polymerase promoters such as the T7 or SP6 polymerase promoters. Alternatively, antisense cDNA constructs that synthesize antisense RNA constitutively or inducibly can be introduced into cell lines. The potency of antisense oligonucleotides for inhibiting TGF may be enhanced using various methods including 1) addition of polylysine (Leonetti et al., Bioconj. Biochem. 1:149-153 (1990)); 2) encapsulation into antibody targeted liposomes (Leonetti et al., Proc. Natl. Acad. Sci. USA 87:2448-2451 (1990) and Zelphati et al., Antisense Research and Development 3:323-338 (1993)); 3) nanoparticles (Rajaonarivony et al., J. Pharmaceutical Sciences 82:912-917 (1993) and Haensler and Szoka, Bioconj. Chem. 4:372-379 (1993)), 4) the use of cationic acid liposomes (Felgner et al., Proc. Natl. Acad. Sci. USA 84:7413-7417 (1987); Capaccioli et al., Biochem. Biophys. Res. Commun. 197:818-825 (1993); Boutorine and Kostina, Biochimie 75:35-41 (1993); Zhu et al., Science 261:209-211 (1993); Bennett et al., Molec. Pharmac. 41:1023-1033 (1992) and Wagner, Science 280:1510-1513 (1993)); and 5) Sendai virus derived liposomes (Compagnon et al., Exper. Cell Res. 200:333-338 (1992) and Morishita et al., Proc. Natl. Acad. Sci. USA 90:8474-8478 (1993)), to deliver the oligonucleotides into cells. Recent techniques for enhancing delivery include the conjugation of the antisense oligonucleotides to a fusogenic peptide, e.g. derived from an influenza hemagglutinin envelop protein (Bongartz et al., Nucleic Acids Res. 22(22):4681-4688 (1994)).

Additional suitable TGFβ inhibitory agents can be readily obtained using methods known in the art to screen candidate agent molecules for binding to TGFβ, such as assays for detecting the ability of a candidate agent to block binding of radiolabeled human TGFβ to cells such as human mesangial cells. Alternatively, candidate compounds may be tested for the ability to inhibit TGF production by mesangial cells using an enzyme-linked immunosorbent assay (ELISA), for example using the R & D Systems (Minneapolis, Minn.) TGF ELISA assay kit (Cat. No. DB 100) (for methods see, e.g. Uotila et al., J. Immunol. Methods 42:11 (1981)).

Suitable TGFβ-specific inhibitory agents can also be developed by known drug design methods, e.g. using structural analysis of the TGFβ molecule employing methods established in the art, for example, using X-ray crystallography to analyze the structure of the complex formed by TGFβ and one of its known inhibitors (see, e.g. Sielecki et al., supra; Rahuel et al., supra, Badasso et al., supra and Dhanaraj et al., supra.), and/or by modifying known TGF antagonists i.e. “lead compounds,” to obtain more potent inhibitors and compounds for different modes of administration (i.e. oral vs. intravenous) (see, e.g. Wexler et al., Amer. J. Hyper. 5:209S-220S (1992)-development of AII receptor antagonists from Losartan.™). For such procedures large quantities of TGFβ can be generated using recombinant technology or purchased commercially (R & D Systems).

The total dosage of alpha-smooth muscle inhibitor or inducer administered to a patient will be determined based upon the particular condition being treated, the route of administration and the treatment of objective. A typical daily dose of inhibitor or inducer administered to a patient will, depending upon the agent used, be between 1 ug and 10 mg. Topical, intranasal and locally injected preparations will, typically, also fall within this range. These dosages are simply guidelines and the actual dosage selected for an individual patient will be determined by the attending physician based upon clinical conditions and using methods well known in the art. Agents may be provided in either a single or multiple dosage regimens and may be given either alone or in conjunction with other therapeutic agents.

The present invention is compatible with any route of administration and any dosage form. Depending upon the particular condition being treated, certain dosage forms will tend to be more convenient or more effective than others. For example, local injection will be the preferred route of administration for accomplishing in vivo ligament repair whereas topical administration will generally be preferred in treating skin cancers and other skin conditions. Apart from parenteral and topical preparations, agents may be administered orally, perorally, internally, intra nasally, rectally, vaginally, lingually, and transdermally. Specific dosage forms include tablets, pills, capsules, powders, aerosols, suppositories, skin patches, parenterals and oral liquids including suspensions, solutions and emulsions. Sustained release dosage forms may also be used. Sustained release may be achieved through the use of an implantable pump, a drug delivery depot of various sizes or shapes, or a solid implant of the therapeutic formulated for temporal release. All dosage forms may be prepared using methods that are standard in the art (see e.g., Remington's Pharmaceutical Sciences, 16th, Ed. A. Oslo Editor, Easton, Pa. (1980).

Inhibitors and inducers of alpha-smooth muscle actin may be used in conjunction with any of the vehicles and excipients commonly employed in pharmaceutical preparations, e.g., talc, gum arabic, lactose, starch, magnesium sterate, cocoa butter, aqueous or non-aqueous solvents, oils, paraffin derivatives, glycols, etc. Coloring and flavoring agents may also be added to preparations, particularly those for oral administration. Solutions can be prepared using water or physiologically compatible organic solvents such as ethanol, 1,2-propylene glycol, polyglycols, dimethyl sulfoxide, fatty alcohols, triglycerides, partial esters of glycerine and the like. Parenteral compositions may be used for intravenous, intraarterial, intramuscular, intraperitoneal, intracutaneous or subcutaneous delivery. These preparations can be made using conventional techniques and may include sterile isotonic saline, water, 1,3-butanediol, ethanol, 1,2-propylene glycol, polyglycols nixed with water, Ringers' solution, etc.

The most effective mode of administration and dosage regimen for the TGFβ inhibitory and ECM degrading agents for use in the methods of the present invention depend on the extent of TGFβ overproduction, the severity of the accumulation of extracellular matrix and resulting impairment of tissue or organ function, the subject's health, previous medical history, age, weight, height, sex and response to treatment and the judgment of the treating physician. Therefore, the amount of TGFβ inhibitory and ECM degrading agents to be administered, as well as the number and timing of subsequent administrations, are determined, by a medical professional conducting therapy based on the response of the individual subject. Initially, such parameters are readily determined by skilled practitioners using appropriate testing in animal models for safety and efficacy, and in human subjects during clinical trials of candidate therapeutic formulations. Suitable animal models of human fibrotic conditions are known (see, e.g. Border and Noble, New Eng. J. Med. 331:1286-1292 (1994), incorporated by reference herein).

After administration, the efficacy of the therapy using the methods of the invention is assessed by various methods including biopsy of kidney, lung or liver or other tissue to detect the amount of extracellular matrix accumulated. An absence of significant excess accumulation of ECM, or a decrease in the amount or expansion of ECM in the tissue or organ will indicate the desired therapeutic response in the subject. Preferably, a non-invasive procedure is used to detect a therapeutic response. For example, changes in TGFβ activity can be measured in plasma samples taken before and after treatment with an inhibitor (see, Eltayeb et al., J. Am. Soc. Nephrol. 8:110A (1997)), and biopsy tissue can be used to individually isolate diseased glomeruli which are then used for RNA isolation. mRNA transcripts for TGFβ, and extracellular matrix components (e.g. collagen) are then determined using reverse transcriptase-polymerase chain reaction (RT-PCR) (Peten et al., J. Exp. Med. 176:1571-1576(1992)).

In the methods of the invention, the TGFβ inhibitory agents are administered concurrently or sequentially. For example, an anti-TGFβ antibody is administered with an anti-renin agent. The inhibitory agents will localize at sites of TGFβ overproduction, e.g. organs such as the kidneys. The inhibitory agents may be labeled, using known radiolabelling methods to detect their localization in a subject after administration. The agents may also be conjugated to targeting molecules such as antibodies to ECM components to improve localization of the agents after administration to the sites of TGFβ overproduction and/or excess accumulation of ECM in a subject.

In another embodiment of the methods of the invention, TGFβ inhibitory agents are administered concurrently or sequentially with at least one agent that degrades accumulated ECM, for example, a serine protease such as plasmin. Alternatively, an agent that induces protease production, such as tPA, is administered to increase protease production at the site(s) of accumulated ECM. tPA binds fibrin (Rondeau et al., Clinical Nephrol. 33:55-60 (1990)) and thus will localize in fibrotic areas where the increased protease production is desired.

In addition to the use of molecules such as antibodies and purified compound such as decorin, nucleic acid encoding the TGFβ inhibitory agents and nucleic acid encoding the agent to directly or indirectly degrade accumulated ECM, are administered to the subject to permit the agents to be expressed and secreted, for inhibiting TGFβ and degrading accumulated ECM. The nucleic acid may be introduced into cells in the subject, for example using a suitable delivery vehicle such as an expression vector or encapsulation unit such as a liposome, or may be introduced directly through the skin, for example in a DNA vaccine.

Alternatively, the nucleic acids encoding the agents are introduced into a cell ex vivo and the cells expressing the nucleic acids are introduced into a subject, e.g. by implantation procedures, to deliver the agents in vivo. Multiple agents can be introduced into a delivery vehicle or in separate vehicles.

Inhibitors and inducers of cell contraction can also be used in conjunction with matrices employed as implants to facilitate tissue healing and as scaffolds to be seeded with cells in vitro for subsequent implantation. In these cases, the inhibitors and inducers can be adsorbed by the matrix and, in some cases, chemically coupled to the matrix.

As can be seen, the present invention provides for the use and delivery of a therapeutic agent to block tissue growth factor beta (TGFβ) and/or smooth muscle actin to treat and prevent such tissue scarring in order to promote natural healing and faster recovery.

In the foregoing description, the method and apparatus of the present invention have been described with reference to a number of examples that are not to be considered limiting. Rather, it is to be understood and expected that variations in the principles of the method and apparatus herein disclosed may be made by one skilled in the art and it is intended that such modifications, changes, and/or substitutions are to be included within the scope of the present invention as set forth in the appended claims. The specification is accordingly to be regarded in an illustrative rather than in a restrictive sense.

What is claimed is:

1. A method of reducing scarring at a particular body site resulting from repair of body tissue at that site, the method comprising the steps of:
determining an amount of a therapeutic agent which will have an intended reduction in scarring; and
administering the therapeutic agent at the particular body site to control at least one of muscle contraction and excessive cell matrix formation.

2. The method according to claim 1, wherein the therapeutic agent inhibits muscle contraction.

3. The method according to claim 1, wherein the therapeutic agent promotes muscle contraction.

4. The method according to claim 2, wherein the therapeutic agent is a smooth muscle actin (SMA) inhibitor.

5. The method according to claim 4, wherein the SMA inhibitor is at least one of PDGF and interferon.

6. The method according to claim 3, wherein the therapeutic agent is a smooth muscle actin (SMA) inducer.

7. The method according to claim 6, wherein the SMA inducer is TGF-β.

8. The method according to claim 7, wherein the TGF-β is administered in an amount from 100 ng/ml to 500 ug/ml.

9. The method according to claim 1, further comprising the steps of:
alternating administration of the therapeutic agent between one of a muscle contraction inducer and one of a muscle contraction inhibitor.

10. The method according to claim 1, wherein the therapeutic agent is at least one of a TGFβ inhibitory agent and a TGFβ-specific inhibitory agent.

11. The method according to claim 1, wherein the amount of the therapeutic agent is a daily dose between 1 ug and 10 mg.

12. The method according to claim 1, wherein the amount of the therapeutic agent is administered in at least one of a form of a tablet, pill, capsule, powder, aerosol, suppository, skin patch, implantable pump, implantable depot, parenteral and an oral liquid including one of a suspension, solution and emulsion.

13. The method according to claim 1, wherein the amount of the therapeutic agent may be used in conjunction with at least one of a talc, gum arabic, lactose, starch, magnesium sterate, cocoa butter, aqueous or non-aqueous solvent, oil, paraffin derivative, and glycol.

14. A method of producing a therapeutic capable of reducing scarring, the method comprising the steps of:
determining an amount of a therapeutic agent to control at least one of muscle contraction and excessive cell matrix formation; and
incorporating the therapeutic agent into a pharmaceutical for administering at a particular body site which will have an intended reduction in scarring.

15. The method according to claim 14, wherein the therapeutic agent inhibits muscle contraction.

16. The method according to claim 14, wherein the therapeutic agent promotes muscle contraction.

17. The method according to claim 15, wherein the therapeutic agent is a smooth muscle actin (SMA) inhibitor.

18. The method according to claim 17, wherein the SMA inhibitor is at least one of PDGF and interferon.

19. The method according to claim 16, wherein the therapeutic agent is a smooth muscle actin (SMA) inducer.

20. The method according to claim 18, wherein the SMA inducer is TGF-β.

21. The method according to claim 20, wherein the TGF-β is administered in an amount from 100 ng/ml to 500 ug/ml.

22. The method according to claim 14, further comprising the steps of:
alternating administration of the therapeutic agent between one of a muscle contraction inducer and one of a muscle contraction inhibitor.

23. The method according to claim 14, wherein the therapeutic agent is at least one of a TGFβ inhibitory agent and a TGF-specific inhibitory agent.

24. The method according to claim 14, wherein the amount of the therapeutic agent is a daily dose between 1 ug and 10 mg.

25. The method according to claim 14, wherein the amount of the therapeutic agent is administered in at least one of a form of a tablet, pill, capsule, powder, aerosol, suppository, skin patch, implantable pump, implantable depot, parenteral and an oral liquid including one of a suspension, solution and emulsion.

26. The method according to claim 14, wherein the amount of the therapeutic agent may be used in conjunction with at least one of a talc, gum arabic, lactose, starch, magnesium sterate, cocoa butter, aqueous or non-aqueous solvent, oil, paraffin derivative, and glycol.
